(19) United States
(12) Patent Application Publication
(10) Pub. No.: US 2007/0265605 A1
(43) Pub. Date: Nov. 15, 2007

(54) Apparatus and method for treating dental tissue

(75) Inventors: Boris Vaynberg, Binyamina (IL); Yotam Zimmerman, Hadera (IL)

Correspondence Address: DEKEL PATENT LTD., DAVID KLEIN, BEIT HAROF'IM, 18 MENUHA VENAHALA STREET, ROOM 27, REHOVOT, 76209, IL

(73) Assignee: Light Instruments Ltd.

(21) Appl. No.: 11/433,514

(22) Filed: May 15, 2006

(51) Int. Cl. A61B 18/18

(52) U.S. Cl. 606/13

(57) ABSTRACT

Apparatus including a pulsed laser operative to deliver laser pulses capable of dental tissue ablation, a liquid cooling system operative to deliver a liquid coolant to a site of the dental tissue ablation, and a controller operative to control and coordinate operation of the pulsed laser and the liquid cooling system such that the liquid coolant is delivered to the site of the dental tissue ablation during a time period that does not overlap in time with delivery of the laser pulses to the site of the dental tissue ablation.

### FIELD OF THE INVENTION

The present invention generally relates to an apparatus and method for removing plaque and decay tissue, dental caries, etc. using laser radiation and liquid cooling, and more particularly to an apparatus using infrared radiation and optimized cooling to ablate efficiently a desired material from a tooth without generating excess heat at the target site or surrounding areas.

### BACKGROUND OF THE INVENTION

Conventional drilling machines (rotary instrument) for treating dental caries can be inaccurate and painful. Pulsed infra red (IR) laser beams can be used instead of mechanical drills to remove hard tissues. However, it is known that lasers produce residual heat in the tooth which may lead to pulp overheat and damage. Excess heat may also produce cracks in the tooth surface.

To overcome the thermal damage problem, current laser systems use water spray simultaneously with laser radiation in a similar way as in mechanical instruments. The water spray is also very useful for removing debris remaining from previous pulses which block the laser radiation and decrease the ablation efficiency.

The problematic side of using water spray is that water strongly absorbs IR laser energy. The more water that is used for cooling, the more light is absorbed by the coolant and ablation efficiency is decreased. This starts to be significant especially in the case of low energy per pulse regimes and in the case of deep cavities in the tooth which may be filled by water. The water spray itself in the air gap between the laser output and the tissue may also partially block the laser beam. The handpieces operating with the tips may be less sensitive because the air gap is shorter, but at some time the spray may create a large water drop on the tip.

A CO₂ (9.6 μm wavelength) laser has been proposed to drill teeth. As long as the spray drops are smaller than the laser wavelength, the laser beam penetrates without absorption by the drops. For the CO₂ laser it is difficult but feasible to produce drops smaller than 9.6 μm. However, for erbium lasers the wavelength is ˜3 μm and it is much harder to produce water drops smaller than 3 μm.

Erbium lasers ablate tissue by the water fraction in the tissue absorbing the energy. There is no selectivity between the intact tissue (enamel, dentine) and the caries. The only difference between the tissues is percentage of the water fraction. The water content in the caries is higher than in dentin and enamel. Thus, if it is desired to remove caries selectively, low energies should be used. The energy may be set lower than the ablation threshold in the intact tissue and above the ablation threshold of caries. However, in light of the above, selective caries removal processes exclude the use of a high volume water spray to achieve effective cooling.

Other lasers that use different mechanisms of ablation or selective tissue removal have been developed. For example, the second harmonic of Alexandrite or third harmonic of the Nd:YAG lasers have been used. These wavelengths are absorbed by caries and do not touch the enamel. The disadvantage of such lasers is high complexity, high cost and no flexible delivery system.

### SUMMARY OF THE INVENTION

The present invention seeks to provide an apparatus and method for treating dental tissue using laser irradiation together with a cooling system which does not influence the ablation speed and increases the cooling efficiency.

An apparatus is provided for treating a target area of a tooth. The apparatus generally comprises a coolant delivery system and a laser (e.g., an IR laser). The coolant delivery system is timed to deliver coolant to the target area that does not overlap in time with the laser pulse. The interleaved timing of the laser pulse and coolant cause ablation of a desired material without generating excess heat which may char surface tissue or cause thermal damage.

The amount of water may be optimized for any laser parameters to provide effective cooling of the tissue and to remove debris left from previous pulses. The amount of cooling water used can be quite large without adversely affecting the ablation, because the water is not delivered during the ablation pulse. Thus, the cooling water can be increased without decreasing the efficiency of the ablation. An air flow may be provided during the application of the laser pulse to help blow water away from the cavity and the tip of the instrument.

There is thus provided in accordance with an embodiment of the present invention apparatus including a pulsed laser operative to deliver laser pulses capable of dental tissue ablation, a liquid cooling system operative to deliver a liquid coolant to a site of the dental tissue ablation, and a controller operative to control and coordinate operation of the pulsed laser and the liquid cooling system such that the liquid coolant is delivered to the site of the dental tissue ablation during a time period that does not overlap in time with delivery of the laser pulses to the site of the dental tissue ablation. The liquid coolant may be water, for example. The laser may be an IR laser, e.g., an erbium laser.

The controller may control delivery of the liquid coolant by means of a solenoid-operated valve, for example.

In accordance with an embodiment of the invention, a source of air pressure may be provided that provides a flow of air to the site of the dental tissue ablation. The controller may control delivery of the flow of air such that the flow of air is provided to the site of the dental tissue ablation during application of the laser pulse to the site.

In accordance with an embodiment of the invention, the liquid cooling system may deliver the liquid coolant to the site of the dental tissue ablation in a pulsed or constant manner.

In accordance with an embodiment of the invention, the controller may control delivery of the liquid coolant such that a time delay exists between shutting off delivery of the liquid coolant and commencement of laser pulse energy delivery to the site of the dental tissue ablation.

There is also provided in accordance with an embodiment of the invention a method including delivering laser pulses to a site of the dental tissue ablation, the laser pulses being capable of dental tissue ablation, and delivering a liquid coolant to the site of the dental tissue ablation during a time period that does not overlap in time with delivery of the laser pulses to the site of the dental tissue ablation.

### BRIEF DESCRIPTION OF THE DRAWINGS

The present invention will be understood and appreciated more fully from the following detailed description, taken in conjunction with the drawings in which:

FIGS. 1 and 2 are simplified pictorial and block diagram illustrations, respectively, of apparatus for treating dental tissue, constructed and operative in accordance with an embodiment of the invention; and

FIG. 3 is a simplified timing diagram that illustrates the timing of the laser pulse and coolant delivery relative to one another in accordance with an embodiment of the invention.

### DETAILED DESCRIPTION OF EMBODIMENTS

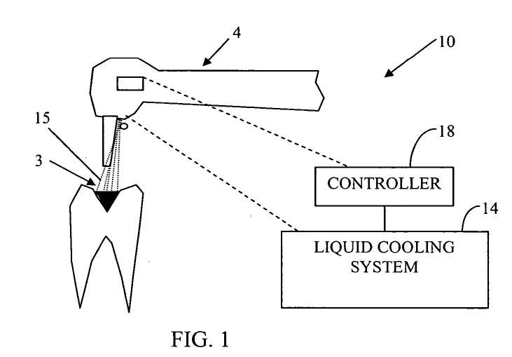

Reference is now made to FIGS. 1 and 2, which illustrate apparatus 10 for treating dental tissue, constructed and operative in accordance with an embodiment of the invention.

Apparatus 10 may include a pulsed laser 12 operative to deliver laser pulses 13 capable of dental tissue ablation. The laser pulses 13 may be delivered to a site 3 of ablation, e.g., a cavity. Laser 12 may be an IR laser, e.g., an erbium laser.

A liquid cooling system 14 may be provided to deliver a liquid coolant 15 (e.g., water) to the site 3 of the dental tissue ablation. The liquid cooling system 14 may include, without limitation, a water tank that feeds water to a handpiece 4 via an on/off solenoid-operated valve 16. For efficient operation (fast switching), the solenoid may be as close as possible to the nozzle of the handpiece 4. The solenoid may be, without limitation, a three way solenoid (with exhaust).

A controller 18 (also referred to as controller unit) may be operatively (e.g., electrically) connected to pulsed laser 12 and liquid cooling system 14 to control and coordinate operation of pulsed laser 12 and liquid cooling system 14 such that the liquid coolant 15 is delivered to the site 3 of the dental tissue ablation during a time period that does not overlap in time with delivery of the laser pulses 13 to the site 3 of the dental tissue ablation. Controller 18, for example, may be in electrical communication (wired or wireless) with solenoid-operated valve 16 and/or a pump (not shown) to control the timed delivery of liquid coolant 15. The liquid cooling system 14 (in conjunction with controller 18) may deliver the liquid coolant 15 in a pulsed or constant manner.

In accordance with an embodiment of the invention, a source of air pressure 20 may be provided that provides a flow of air 17 to the site 3 of the dental tissue ablation. The controller 18 may control source of air pressure 20 such that the flow of air 17 is provided to the site 3 of the dental tissue ablation during application of the laser pulse 13 to the site 3.

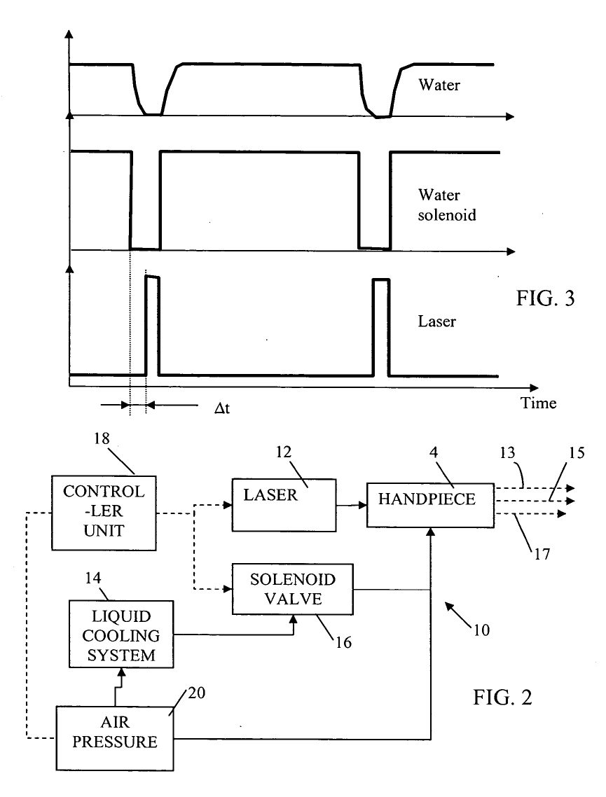

As seen best in FIG. 3, the liquid coolant 15 is delivered during a time period that does not overlap in time with delivery of the laser pulses 13. Controller 18 may insert a time delay Δt between shutting off delivery of the liquid coolant 15 and commencement of laser pulse energy delivery to the site 3 of the dental tissue ablation. This may, for example, allow time for the liquid spray to stop and for the air flow 17 to blow water out of the cavity. The air pressure and volume may be set to high, whereas the water volume may be defined by the on/off cycle of the solenoid. This may allow changing the water amount without influencing the laser screening.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. Apparatus comprising:
a pulsed laser operative to deliver laser pulses capable of dental tissue ablation;
a liquid cooling system operative to deliver a liquid coolant to a site of the dental tissue ablation; and
a controller operative to control and coordinate operation of said pulsed laser and said liquid cooling system such that the liquid coolant is delivered to the site of the dental tissue ablation during a time period that does not overlap in time with delivery of the laser pulses to the site of the dental tissue ablation.

2. The apparatus according to claim 1, wherein said liquid coolant comprises water.

3. The apparatus according to claim 1, wherein said laser comprises an IR laser.

4. The apparatus according to claim 1, wherein said laser comprises an erbium laser.

5. The apparatus according to claim 1, wherein said controller controls delivery of said liquid coolant by means of a solenoid-operated valve.

6. The apparatus according to claim 1, further comprising a source of air pressure adapted to provide a flow of air to the site of the dental tissue ablation.

7. The apparatus according to claim 6, wherein said controller control delivery of the flow of air such that the flow of air is provided to the site of the dental tissue ablation during application of the laser pulse to the site.

8. The apparatus according to claim 1, wherein said liquid cooling system is operative to deliver said liquid coolant to the site of the dental tissue ablation in a pulsed manner.

9. The apparatus according to claim 1, wherein said liquid cooling system is operative to deliver said liquid coolant to the site of the dental tissue ablation in a constant manner.

10. The apparatus according to claim 1, wherein said controller controls delivery of said liquid coolant such that a time delay exists between shutting off delivery of said liquid coolant and commencement of laser pulse energy delivery to the site of the dental tissue ablation.

11. A method comprising:
delivering laser pulses to a site of the dental tissue ablation, the laser pulses being capable of dental tissue ablation; and
delivering a liquid coolant to the site of the dental tissue ablation during a time period that does not overlap in time with delivery of the laser pulses to the site of the dental tissue ablation.

12. The method according to claim 11, wherein said liquid coolant comprises water.

13. The method according to claim 11, wherein said laser comprises an IR laser.

14. The method according to claim 11, wherein said laser comprises an erbium laser.

15. The method according to claim 11, wherein delivery of said liquid coolant is controlled by means of a solenoid-operated valve.

16. The method according to claim 11, further comprising providing a flow of air to the site of the dental tissue ablation.

17. The method according to claim 16, wherein the flow of air is provided to the site of the dental tissue ablation during application of the laser pulse to the site.

18. The method according to claim 11, wherein said liquid coolant is delivered to the site of the dental tissue ablation in a pulsed manner.

19. The method according to claim 11, wherein said liquid coolant is delivered to the site of the dental tissue ablation in a constant manner.

20. The method according to claim 11, wherein a time delay exists between shutting off delivery of said liquid coolant and commencement of laser pulse energy delivery to the site of the dental tissue ablation.
